(19) United States
(12) Patent Application Publication
(10) Pub. No.: US 2025/0352499 A1
(43) Pub. Date: Nov. 20, 2025

(54) NOVEL COMBINATION BASED ON SALIFIED BUTYRIC AND AND YEASTS, COMPOSITIONS CONTAINING IT AND THEIR USE IN THERAPY

(71) Applicant: NYUMA PHARMA SRL, Arona (NO) (IT)

(72) Inventors: Angelo Pizzoni, Arona (NO) (IT); Paolo Pizzoni, Arona (NO) (IT)

(73) Assignee: NYUMA PHARMA SRL, Arona (NO) (IT)

(21) Appl. No.: 18/870,907

(22) PCT Filed: Jun. 14, 2023
(86) PCT No.: PCT/IB2023/056126

§ 371 Date: Dec. 2, 2024

(30) Foreign Application Priority Data
Jun. 15, 2022 (IT) 102022000012695
Nov. 22, 2022 (IT) 102022000024033

(51) Int. Cl. A61K 31/19; A61K 9/00; A61K 9/48; A61K 36/064

(52) U.S. Cl. CPC A61K 31/19; A61K 9/0031; A61K 9/009; A61K 9/4825; A61K 36/064

(57) ABSTRACT

The present invention relates to a novel combination of salified butyric acid and yeast, the pharmaceutical and nutraceutical compositions containing the combination as well as their use in the prevention and treatment of intestinal diseases.

The present invention relates to a novel combination of salified butyric acid and yeast, the pharmaceutical and nutraceutical compositions containing the combination as well as their use in therapy, in particular in the prevention and treatment of intestinal diseases.

### TECHNICAL BACKGROUND

Intestinal diseases, such as colon diseases, include inflammatory bowel diseases, such as Crohn's disease and ulcerative colitis, and non-inflammatory colon diseases such as irritable bowel syndrome (IBS), are widespread functional gastrointestinal disorders. Crohn's disease is an inflammation with segmental appearance present in any part of the bowel from the mouth to the anus, the most affected areas being the ileum or colon or both. It is characterized by the presence of ulcerations alternating with stretches of healthy bowel, which if untreated lead to stenosis. The trend is chronic relapsing. Stenoses that cause a narrowing of the intestinal lumen have a fibrotic component and an inflammatory component (leukocytes and mucosal edema), cause abdominal pain, meteorism and abdominal distension with partial or total occlusion of the lumen resulting in constipation.

Ulcerative colitis is an inflammation of the mucosa of the colon rectum that can spread continuously throughout the colon. It is characterized by chronic inflammation with ulcerative lesions. The trend is alternating acute episodes followed by periods of clinical remission and can result in severe bleeding, colon perforation, severe dehydration, toxic megacolon (acute dilation of the colon), and an increased risk of colon cancer.

IBS (irritable bowel syndrome) is widespread high-functional gastrointestinal disorder characterized by chronic and recurrent abdominal discomfort and pain with alterations to the alvus.

In addition to the above diseases, diverticular disease (DD) is also widespread. DD is a clinical condition, the prevalence (age-dependent) of which is in fact continuously increasing. Along with a symptomatology (abdominal pain and swelling, diarrhea and/or constipation) that overlaps with that of the Irritable Bowel Syndrome (IBS), DD can lead to inflammation/infection of the diverticula, resulting in a clinical picture of acute diverticulitis, which can be complicated or uncomplicated, the management of which is particularly difficult especially in the elderly patient.

Several drugs are available for the treatment of colon diseases.

The “biologic” drugs include monoclonal antibodies synthesized in laboratory and are defined as biologic because they target a specific biological molecule such as TNFα. Among these, Infliximab which is administered intravenously (hospital infusion therapy) whereas Adalimumab is administered subcutaneously (home therapy) are commercially available, and both are approved for moderate-to-severe disease. The limitation of these therapies is that not all patients respond and that a portion of responding patients tends to lose their response over time. Furthermore, these therapies are not free of side effects such as infusion reactions and risk of systemic infections. Other drugs used are antibiotics that have the well-known side effects, anti-diarrhea drugs (loperamide) that have to be used with great caution because they can increase the risk of toxic megacolon, and pain relievers that are basically just to relieve pain and discomfort.

Regarding DD, since dysbiosis (and in particular Small Intestine Bacterial Overgrowth—SIBO) represents the alteration of intestinal micro-ecology that is frequently associated with diverticular disease, the use of therapies capable of modifying the microbiota (systemic and topical antibiotics, probiotics and pre-biotics as well as post-biotics) is currently the “standard of care”. But unfortunately, most probiotics are antibiotic-sensitive and therefore are destroyed by the concurrent administration of antibiotics.

In general, if the drug therapy, diet and lifestyle are unable to alleviate the signs and symptoms, and complications occur, surgery is usually recommended.

Butyric acid, and particularly its salts, is also used in colon diseases and exerts many useful functions, for example, it regulates the intestinal alvus, decreases intestinal pH, acts as a natural antidiarrheal, is an energy source for colonocytes, increases mucus production and also has anti-inflammatory activity.

However, despite the various drugs on the market, there is a need for novel therapeutic approaches, for example combination therapy, which allows to more comprehensively treat intestinal diseases, acting through different mechanisms of action.

### OBJECTS OF THE INVENTION

A first object of the present invention is to provide a novel combination of salified butyric acid with at least one probiotic yeast and its use in therapy, in particular in the prevention and treatment of intestinal diseases such as colon diseases.

Another object of the present invention is to provide a pharmaceutical and/or nutraceutical composition containing the combination of the invention and its use in therapy, in particular in the prevention and treatment of intestinal diseases such as colon diseases.

Finally, a further object of the present invention is to provide a process for the preparation of the compositions of the invention.

### DESCRIPTION OF THE INVENTION

According to one of its aspects, subject-matter of the invention is a combination consisting of a pharmaceutically/physiologically acceptable salt of butyric acid and at least one yeast selected from the Saccharomyces and Kluyveromyces species.

Butyric acid (or butanoic acid) has the formula CH₃—CH₂—CH₂—COOH and is a colorless oily liquid at room pressure and temperature.

Pharmaceutically/physiologically acceptable salts of butyric acid, e.g. calcium salt and sodium salt, are known and commercially available. According to a preferred embodiment, the butyric acid salt in the combination of the invention is sodium salt. Even where not expressly stated, the butyric acid salts of the invention are always pharmaceutically/physiologically acceptable.

According to the invention, the at least one yeast is preferably a probiotic yeast selected from the Saccharomyces and Kluyveromyces species, preferably is selected from Saccharomyces cerevisiae, Saccharomyces boulardii, Kluyveromyces marxianus and mixtures thereof.

According to a preferred embodiment, said at least one yeast is the Kluyveromyces marxianus B0399 strain, which is known and commercially available.

According to another preferred embodiment, said at least one yeast is selected from Saccharomyces boulardii.

Preferably, said pharmaceutically/physiologically acceptable salt of butyric acid is (micro)encapsulated but it's also possible to use said salt in non-(micro)encapsulated form.

According to a preferred embodiment, said at least one yeast is in freeze-dried form.

According to a preferred embodiment, the combination of the invention is constituted by a pharmaceutically/physiologically acceptable salt of (micro)encapsulated butyric acid, preferably sodium salt, and Kluyveromyces marxianus B0399 yeast.

Preferably, said pharmaceutically/physiologically acceptable salt of butyric acid is (micro)encapsulated and released into the bowel.

Processes are known for the (micro)encapsulation of substances for their administration and release in the bowel. Any encapsulation technique can be used to obtain the (micro)encapsulated salt of butyric acid for the invention.

For its administration to a subject in the need thereof, the combination of the invention is preferably formulated in a pharmaceutical composition.

According to another of its aspects, subject-matter of the invention is a pharmaceutical and/or nutraceutical composition comprising the combination of the invention, in all of its forms set forth above, and possibly one or more pharmaceutically acceptable excipients.

According to a preferred embodiment, the composition of the invention is suitable for the oral or rectal administration.

According to a preferred embodiment, the combination is contained in oral solid compositions of the capsule, sachet or tablet type, preferably a capsule.

According to another preferred embodiment, the combination is contained in compositions for rectal use, such as micro-enemas, suppositories and the like.

The compositions of the invention can be defined as pharmaceutical and/or nutraceutical compositions.

For the preparation of the preferred solid oral composition according to the invention, for example a capsule, tablet or granule to be packaged in sachets, it is possible to use the methods known in the art.

According to a particularly preferred embodiment, the oral compositions of the invention are in oral form, or form of capsules or sachets, containing the pharmaceutically/physiologically acceptable salt of butyric acid, preferably sodium salt, in micro-encapsulated form and the at least one yeast, preferably the Kluyveromyces marxianus B0399 strain, in freeze-dried form, possibly together with pharmaceutically acceptable carriers and excipients.

According to another embodiment, the oral compositions of the invention are in oral form, form of capsules or sachets, containing the pharmaceutically/physiologically acceptable salt of butyric acid, preferably sodium salt, in micro-encapsulated form and the at least one yeast, preferably a Saccharomyces strain, for example a Saccharomyces boulardii strain, in freeze-dried form, possibly together with pharmaceutically acceptable carriers and excipients.

According to a particularly preferred embodiment, the rectal compositions of the invention are in the form of micro-enemas containing the pharmaceutically/physiologically acceptable salt of butyric acid, preferably sodium salt, in (micro)encapsulated form and the at least one yeast, preferably the Kluyveromyces marxianus B0399 strain or a Saccharomyces strain, for example Saccharomyces boulardii, in freeze-dried form, possibly together with pharmaceutically acceptable carriers and excipients conventionally used in the preparation of these formulations.

The compositions for oral or rectal administration can be prepared according to the methods known in the art.

The compositions of the invention can contain 100 to 1,000 mg pharmaceutically/physiologically acceptable salt of butyric acid, preferably sodium salt, preferably 150 to 500 mg, more preferably about 200 mg sodium butyrate.

Alternatively, the compositions of the invention can contain 500 to 2,000 mg pharmaceutically/physiologically acceptable salt of butyric acid, preferably sodium salt, preferably 600 to 1,500 mg, more preferably 600 to 700 mg sodium butyrate.

The skilled in the art is perfectly capable of calculating the amount of product to put into the composition in case the titre of the butyrate salt is not 100%.

The compositions of the invention can contain 5 to 20 million CFUs, for example, 5 to 15 million CFUs (colony-forming units) of yeast according to the invention, preferably Kluyveromyces marxianus, preferably the B0399 strain, preferably about 10 to 20 million CFUs.

Alternatively, the compositions of the invention can contain 5 to 20 million CFUs, for example, 5 to 15 billion CFUs (colony-forming units) of Saccharomyces, preferably Saccharomyces cerevisiae or boulardii, preferably about 10 to 20 million CFUs.

The compositions of the invention are administered once or more times a day and the dosages can vary according to the weight and age of the subject to be treated, as well as the type and severity of the disease.

The “subject” according to the invention is a mammal, preferably, but not only, the human being.

According to another of its aspects, subject-matter of the invention is the combination of the invention for its use in therapy, in particular in the treatment and prevention of intestinal diseases, for example but not only colon diseases.

According to another of its aspects, subject-matter of the invention is the composition of the invention for its use in therapy, in particular in the treatment and prevention of intestinal diseases, for example but not only colon diseases.

According to another of its aspects, subject-matter of the invention is a method for the treatment and prevention of intestinal diseases, for example but not only colon diseases, which comprises administering an effective amount of the combination or composition of the invention to a subject in the need thereof.

By the term “colon diseases” is meant herein to include inflammatory bowel diseases, such as but not limited to Crohn's disease and ulcerative colitis, irritable bowel syndrome, dysbioses, colitis and the like.

By the term “colon diseases” is meant herein to include diverticular disease (DD) as well.

(Micro)encapsulated sodium butyrate (and thus able to release the active ingredient at the colic level), in addition to improving the IBS-like symptomatology of DD, is capable of reducing the incidence of acute diverticulitis episodes and improve the quality of life of DD patients.

The combination of the invention thus is a novel and effective first-choice treatment of diverticular disease.

According to another of its aspects, subject-matter of the invention is a kit comprising compositions containing a pharmaceutically/physiologically acceptable salt of butyric acid, preferably sodium salt, preferably encapsulated as described above, and compositions containing at least one yeast selected from the Saccharomyces and Kluyveromyces species, preferably in freeze-dried form.

The compositions contained in the kit of the invention can contain suitable pharmaceutically acceptable excipients and carriers.

According to an embodiment, the kit comprises 1 to 100 compositions comprising a pharmaceutically acceptable salt of butyric acid, preferably sodium salt, and 1 to 100 compositions comprising the yeast as defined herein, preferably Kluyveromyces marxianus, preferably the B0399 strain; the kit can preferably comprise 7 to 50, preferably 10 to 30 of each composition, the number of the two different compositions being preferably the same.

The preferred embodiments described above for the combination and compositions containing the combination also apply to the compositions of the kit.

The compositions of the kit can be taken simultaneously or otherwise within a short time frame.

The compositions of the kit are preferably packaged together with a leaflet.

The invention will be now described in more detail in the Experimental Section below, by way of illustration only and in no way limiting.

### EXPERIMENTAL SECTION

### Example 1

A composition is prepared in capsules of rigid gelatin for oral use containing
20 million CFUs of Kluyveromyces marxianus B0399 in freeze-dried form;
550 mg sodium salt of (micro)encapsulated butyric acid, containing 200 mg butyric acid (active ingredient) sodium salt
   together with conventional carries and excipients.

### Example 2

A composition is prepared in capsules of rigid gelatin of vegetable origin for oral use containing
20 million CFUs of Kluyveromyces marxianus B0399 in freeze-dried form;
500 mg sodium salt of (micro)encapsulated butyric acid, containing 200 mg butyric acid (active ingredient) sodium salt
   together with conventional carries and excipients.

### Example 3

A composition is prepared in capsules of rigid gelatin of vegetable origin for oral use containing
20 million CFUs of Kluyveromyces marxianus B0399 in freeze-dried form;
600-700 mg calcium salt of (micro)encapsulated butyric acid
   together with conventional carries and excipients.

### Example 4

A composition is prepared in soft gel capsules for oral use containing
20 million CFUs of Kluyveromyces marxianus B0399 in freeze-dried form;
550 mg sodium salt of (micro)encapsulated butyric acid containing 200 mg butyric acid (active ingredient) sodium salt
   together with conventional carries and excipients.

### Example 5

A composition is prepared in micro-enemas for rectal use containing
20 million CFUs of Kluyveromyces marxianus B0399 in freeze-dried form;
550 mg sodium salt of (micro)encapsulated butyric acid containing 200 mg butyric acid (active ingredient) sodium salt
   together with conventional carries and excipients.

### Example 6

A composition is prepared in capsules of rigid gelatin for oral use containing
10 million CFUs of Kluyveromyces marxianus B0399 in freeze-dried form;
670 mg sodium salt of micro-encapsulated butyric acid
   together with conventional carries and excipients.

### Example 7

A composition is prepared in capsules of rigid gelatin of vegetable origin for oral use containing
10 million CFUs of Kluyveromyces marxianus B0399 in freeze-dried form;
670 mg calcium salt of micro-encapsulated butyric acid
   together with conventional carries and excipients.

### Example 8

A composition is prepared in soft gel capsules for oral use containing
10 million CFUs of Kluyveromyces marxianus B0399 in freeze-dried form;
670 mg sodium salt of micro-encapsulated butyric acid
   together with conventional carries and excipients.

### Example 9

A composition is prepared in capsules of rigid gelatin of vegetable origin for oral use containing
10 million CFUs of Kluyveromyces marxianus B0399 in freeze-dried form;
600-700 mg calcium salt of micro-encapsulated butyric acid
   together with conventional carries and excipients.

### Example 10

A composition is prepared in soft gel capsules for oral use containing
10 million CFUs of Kluyveromyces marxianus B0399 in freeze-dried form;
600-700 mg sodium salt of micro-encapsulated butyric acid
   together with conventional carries and excipients.

### Example 11

A composition is prepared in micro-enemas for rectal use containing
10 million CFUs of Kluyveromyces marxianus B0399 in freeze-dried form;
600-700 mg sodium salt of micro-encapsulated butyric acid
   together with conventional carries and excipients.

### Example 12

A composition is prepared in capsules of rigid gelatin for oral use containing
10 million CFUs of Kluyveromyces marxianus B0399 in freeze-dried form;
550 mg sodium salt of (micro)encapsulated butyric acid, containing 200 mg butyric acid (active ingredient) sodium salt
   together with conventional carries and excipients.

### Example 13

A composition is prepared in capsules of rigid gelatin of vegetable origin for oral use containing
10 million CFUs of Kluyveromyces marxianus B0399 in freeze-dried form;
500 mg sodium salt of (micro)encapsulated butyric acid, containing 200 mg butyric acid (active ingredient) sodium salt
   together with conventional carries and excipients.

### Example 14

A composition is prepared in capsules of rigid gelatin of vegetable origin for oral use containing
10 million CFUs of Kluyveromyces marxianus B0399 in freeze-dried form;
600-700 mg calcium salt of (micro)encapsulated butyric acid
   together with conventional carries and excipients.

### Example 15

A composition is prepared in soft gel capsules for oral use containing
10 million CFUs of Kluyveromyces marxianus B0399 in freeze-dried form;
550 mg sodium salt of (micro)encapsulated butyric acid containing 200 mg butyric acid (active ingredient) sodium salt
   together with conventional carries and excipients.

### Example 16

A composition is prepared in gelatin capsules for oral use containing
20 million CFUs of Saccharomyces boulardii in freeze-dried form;
550 mg sodium salt of (micro)encapsulated butyric acid, containing 200 mg butyric acid (active ingredient) sodium salt
   together with conventional carries and excipients.

### Example 17

A composition is prepared in sachets for oral use containing
20 million CFUs of Saccharomyces boulardii in freeze-dried form;
550 mg sodium salt of (micro)encapsulated butyric acid, containing 200 mg butyric acid (active ingredient) sodium salt
   together with conventional carries and excipients.

1. A combination consisting of a pharmaceutically/physiologically acceptable salt of butyric acid and at least one yeast selected from Kluyveromyces species.

2. The combination according to claim 1, wherein said at least one yeast is a Kluyveromyces marxianus probiotic yeast.

3. The combination according to claim 1, wherein said butyric acid salt is sodium salt.

4. The combination according to claim 1, wherein said pharmaceutically/physiologically acceptable salt of butyric acid is in encapsulated or micro-encapsulated form.

5. The combination according to claim 1, wherein said Kluyveromyces marxianus yeast is in freeze-dried form.

6. A pharmaceutical or nutraceutical composition comprising the combination according to claim 1, together with one or more pharmaceutically acceptable carriers and/or excipients.

7. The composition according to claim 6, for oral administration.

8. The composition according to claim 7, for rectal administration.

9. The composition according to claim 6, comprising 100 to 1,000 mg pharmaceutically/physiologically acceptable salt of butyric acid.

10. The composition according to claim 6, comprising 5 to 20 million CFUs (colony-forming units) of Kluyveromyces marxianus.

11-14. (canceled)

15. The composition according to claim 7, wherein said composition is in form of capsules or tablets.

16. The composition according to claim 8, wherein said composition is in form of micro-enemas.

17. The composition according to claim 9, wherein said pharmaceutically/physiologically acceptable salt of butyric acid is sodium salt.

18. The composition according to claim 9, comprising 150 to 500 mg of sodium butyrate.

19. The composition according to claim 9, comprising about 200 mg of sodium butyrate.
